(19) United States
(12) Patent Application Publication
(10) Pub. No.: US 2015/0282745 A1
(43) Pub. Date: Oct. 8, 2015

(54) Measuring Device for an Analyte

(71) Applicant: Bionime Corporation, Taichung City (TW)

(72) Inventors: Cheng-Teng HSU, Taichung City (TW); Hsi-Wen HUANG, Taichung City (TW)

(21) Appl. No.: 14/539,123

(22) Filed: Nov. 12, 2014

(30) Foreign Application Priority Data
Apr. 8, 2014 (TW) 103112911

(51) Int. Cl. A61B 5/145; A61B 5/00

(52) U.S. Cl. CPC A61B 5/14532; A61B 5/002; A61B 5/68

(57) ABSTRACT

A measuring device for an analyte in a specimen includes a device body, and a sensing unit and an installation unit that are disposed on the device body. The sensing unit includes a measuring module to measure a parameter of the analyte in the specimen. The installation unit removably installs the device body on a drug injection device.

### CROSS-REFERENCE TO RELATED APPLICATION

This application claims priority to Taiwanese Application No. 103112911, filed on Apr. 8, 2014.

### BACKGROUND OF THE INVENTION

1. Field of the Invention

The invention relates to a measuring device, and more particularly to a measuring device adapted for an analyte and to be attached to a drug injection device.

2. Description of the Related Art

Most diabetic patients inject insulin to him/herself for blood glucose adjustment. However, time and a dose of the insulin injection are significantly associated with the blood glucose level at the moment. As a result, how to promote convenience of measuring the blood glucose level is a goal of current researches.

Conventional blood glucose measuring devices integrated with an insulin pump, such as those disclosed in U.S. Pat. No. 8,460,243, US patent application publication no. 20100099973 and WO patent publication no. 2000013580, are unable to set additional variable factors (e.g., exercises that may result in lower blood glucose level), and are unable to handle unexpected behaviors of the patient, thereby causing inconvenience in use.

Another kind of conventional blood glucose measuring devices with integrated insulin pen into a single unit, such as those disclosed in US patent application publication nos. 20120238853, 20110275986, 20120065487 and 20130245545, are capable of setting functions, but complex structures thereof are unfavorable in manufacturing and in use. Patients may spend a relatively long period of time to be familiar with operation for safe use, thereby causing inconvenience.

### SUMMARY OF THE INVENTION

Therefore, an object of the present invention is to provide a measuring device for an analyte in a specimen that promotes convenience in use.

According to the present invention, a measuring device for an analyte in a specimen comprises:
a device body;
a sensing unit disposed on the device body, and including a measuring module configured to measure a parameter of the analyte in the specimen; and
an installation unit disposed on the device body, and configured to removably install the device body on a drug injection device.

### BRIEF DESCRIPTION OF THE DRAWINGS

Other features and advantages of the present invention will become apparent in the following detailed description of the preferred embodiments with reference to the accompanying drawings, of which:

FIG. 1 is a block diagram illustrating a first preferred embodiment of a measuring device for an analyte according to the present invention, and a drug injection device;

FIG. 2 is a perspective view showing the first preferred embodiment;

FIG. 3 is a perspective view showing the first preferred embodiment in a state of retaining the drug injection device therein;

FIG. 4 is a perspective view showing a second preferred embodiment of the measuring device according to the present invention;

FIG. 5 is a side view showing the second preferred embodiment in a state of retaining the drug injection device;

FIG. 6 is a perspective view showing a third preferred embodiment of the measuring device according to the present invention;

FIG. 7 is a perspective view showing the third preferred embodiment in a state of retaining the drug injection device;

FIG. 8 is a perspective view showing a fourth preferred embodiment of the measuring device according to the present invention, in a state of retaining the drug injection device; and

FIG. 9 is a perspective view showing the fourth preferred embodiment and the drug injection device.

### DETAILED DESCRIPTION OF THE PREFERRED EMBODIMENTS

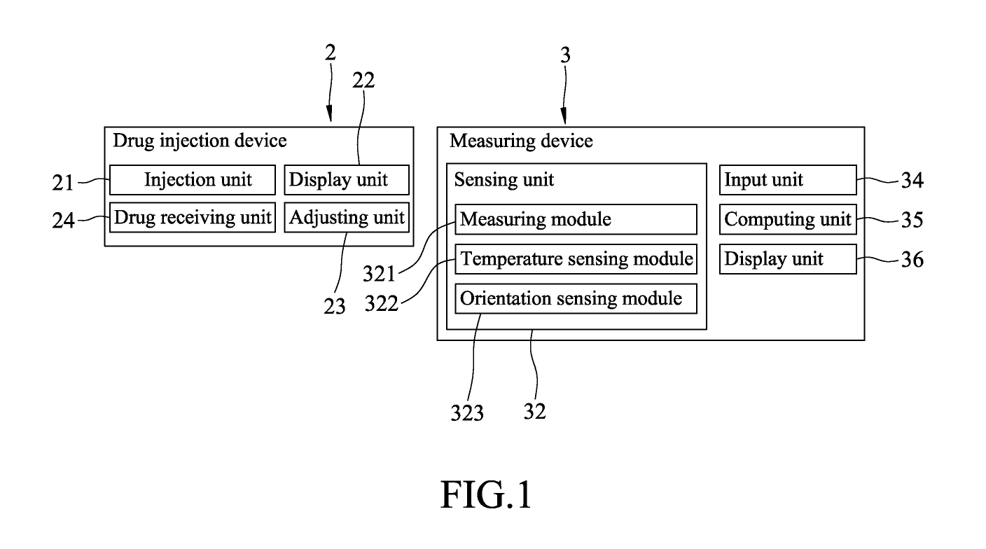

Referring to FIGS. 1 to 3, the first preferred embodiment of the measuring device 3 according to this invention is adapted for an analyte in a specimen, and is to be mounted to a drug injection device 2. In this embodiment, the measuring device 3 is a blood glucose measuring device, and the drug injection device 2 is an insulin injection pen.

The drug injection device 2 includes an injection unit 21, a display unit 22, an adjusting unit 23, and a drug receiving unit 24 for placing a drug therein. The display unit 22 is configured to display at least one of the following information: a dose of the drug that is to be injected, a drug type, a remaining amount of the drug, and whether or not the drug has expired. The adjusting unit 23 is configured for adjusting a to-be-outputted dose of the drug in the drug receiving unit 24. In addition, the drug injection device 2 may be configured with a puncturing structure for collecting a blood sample for analysis and measurement. In this embodiment, the drug used herein is insulin.

The measuring device 3 includes a device body 31, a sensing unit 32, an installation unit 33, an input unit 34, a computing unit 35 and a display unit 36, and may cooperate with a sensing strip 9 to perform measurement for the analyte.

The sensing unit 32 is disposed on the device body 31, and includes a measuring module 321 configured to measure a parameter of the analyte (e.g., the blood glucose) in the specimen (e.g., the blood), a temperature sensing module 322 configured to sense an ambient temperature, and an orientation sensing module 323.

In this embodiment, the sensing strip 9 may be an electrochemical or optical sensing strip, which includes a reaction groove and a reagent layer. The reaction groove is configured for receiving the specimen, and the analyte contained in the specimen reacts with the reagent layer in the reaction groove. After placing the sensing strip 9 in the measuring device 3, the measuring module 321 measures signals of the chemical reaction to obtain a readout value of the analyte. Since such techniques are well known in the art, further details thereof are omitted herein for the sake of brevity.

The temperature sensing module 322 is configured to generate a notification signal when the ambient temperature sensed thereby is out of a preset temperature range. The preset temperature range is determined according to a storage temperature range of the drug. As an example, the insulin may deteriorate if not stored within a temperature range of 2° C. to 30° C. Therefore, a temperature range between 3° C. and 29° C., between 4° C. and 28° C., or other suitable range may be set as the preset temperature range for the insulin. Slightly narrowing the storage temperature range may reduce risk from errors in temperature sensing and obtain better effect in preservation of the drug. Different drugs may have different suitable storage temperature ranges, and the preset temperature range should be determined accordingly.

The orientation sensing module 323 is configured to sense an output direction of the drug injection device 2 that is retained on the measuring device 3, and to generate a notification signal when a drug output part of the drug injection device 2 is not placed in a prime shot direction that is vertical to a ground plane. Furthermore, the display unit 36 may output a display signal for notifying the user to vertically place the drug injection device 2 in such a condition.

The user may put the drug in the drug injection device 2 or in a bag when going out. The temperature sensing module 322 of the measuring device 3 periodically senses the ambient temperature to prevent the drug in the drug injection device 2 from becoming ineffective due to an excessively high or low temperature. As an example, since a diabetic patient usually measures the blood glucose level every five hours, the display unit 36 of the measuring device 3 displays the highest and the lowest temperature values within the last five hours as a reference when the patient intends to measure the blood glucose.

The installation unit 33 is disposed on the device body 31, and is configured to removably install the device body 31 on the drug injection device 2. The installation unit 33 includes a positioning component 330 disposed on the device body 31 and defining a receiving space. The receiving space is configured to permit extension of the drug injection device 2 therein, so as to retain removably the drug injection device 2 on the positioning component 330.

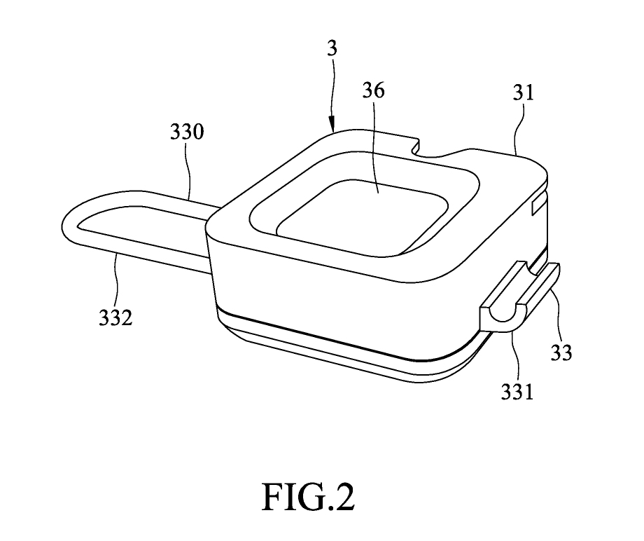

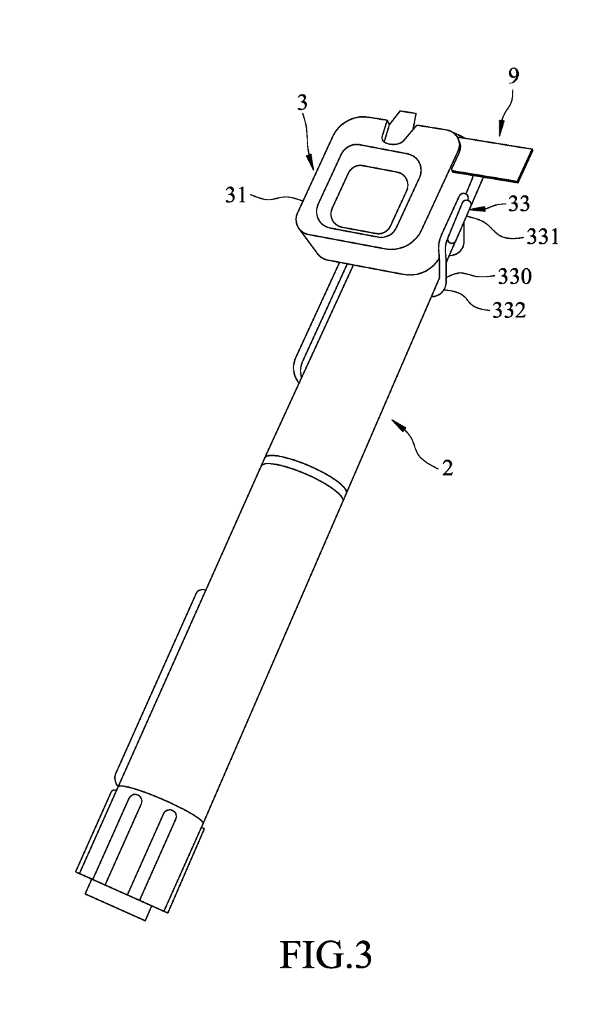

Referring to FIGS. 2 and 3, the positioning component 330 includes a fixing member 331 disposed on an end of the device body 31, and an encircling member 332 that is elastically deformable and that is disposed on another end of the device body 31. The encircling member 332 is removably hooked on the fixing member 331, cooperates with the device body 31 to form the receiving space, and tightly retains the drug injection device 2 on the device body 31 when hooked with the fixing member 331.

Referring to FIGS. 1 to 3, the input unit 34 is disposed on the device body 31, and is operable to input personal data associated with at least one of a height, a weight, sex, an age, a meal time, an amount of carbohydrate, and an amount of exercise.

The computing unit 35 is disposed on the device body 31, receives the personal data inputted from the input unit 34, performs computation according to the personal data, and outputs a computation signal associated with at least one of a recommended dose of the drug, a drug type, and a recommended time of drug injection. Since the feature of the present invention does not reside in computation of the recommended dose of the drug, details thereof are omitted herein for the sake of brevity.

The display unit 36 is disposed on the device body 31, receives the computation signal outputted by the computing unit 35, and outputs a display signal corresponding to the computation signal, as well as indication signals generated by the temperature sensing module 322 and the orientation module 323.

In addition, the indication signals generated by the temperature sensing module 322 and the orientation module 323 may be outputted using a different type of warning devices, such as an acoustic warning device (e.g., a speaker, a buzzer, etc.), a visual warning device (e.g., a screen of light emitting diodes, a screen of liquid crystal, etc.), a tactile warning device (e.g., a vibrator, etc.), a wireless warning device (e.g., a wireless signal transmitter that is configured to wirelessly transmit a message to a doctor or a patient's relative), or other types of the warning devices. However, the present invention should not be limited in this respect.

In use, the encircling component 332 of the measuring device 3 is tightly hooked on the fixing component 331, so that the measuring device 3 hangs on the drug injection device 2, thereby facilitating the user to inspect conditions of the insulin and the blood glucose at any time.

This embodiment of the present invention has the following advantages:

1. By use of the installation unit 33 to removably install the measuring device 3 on the drug injection device 2, this embodiment has a relatively simple structure and is easier to manufacture compared to the conventional techniques, and is compatible for use with conventional drug injection devices 2 sold in the market. Therefore, the user may follow the previous operation of injection, thereby preventing risks from operation error, and establishing a habit of measuring the blood glucose before injection of the insulin, so as to achieve effective and safe injection of the insulin. In addition, integration of the measuring device 3 and the drug injection device 2 may reduce overall weight and enhance portability.

2. By virtue of the temperature sensing module 322, the ambient temperature may be sensed so as to indirectly manage the storage temperature of the drug received in the drug injection device 2 by generating the indication signal when the ambient temperature is out of the preset temperature range. In addition, since the patient (i.e., the user) may measure the blood glucose about every five hours, the display unit 36 of the measuring device 3 is configured to display the highest and the lowest ambient temperature within the last five hours, thereby preventing the patient from using the insulin that may have become ineffective due to an excessively high or low temperature, and enhancing safety in use.

3. The orientation sensing module 323 generates the indication signal when the drug injection device 2 is not placed in the prime shot direction, thereby preventing presence of bubbles in an insulin tank (not shown) or in a syringe needle of the drug injection device 2, and enhancing dose accuracy of the drug and safety in use.

4. The input unit 34, the computing unit 35 and the display unit 36 enable the function of providing information of the dose of the insulin, the type of the insulin, and recommended time of drug injection, thereby enhancing convenience in use.

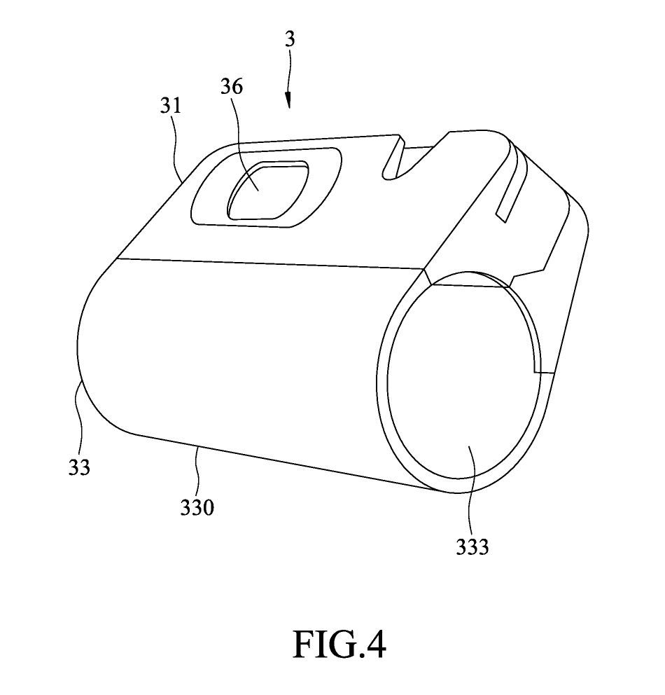

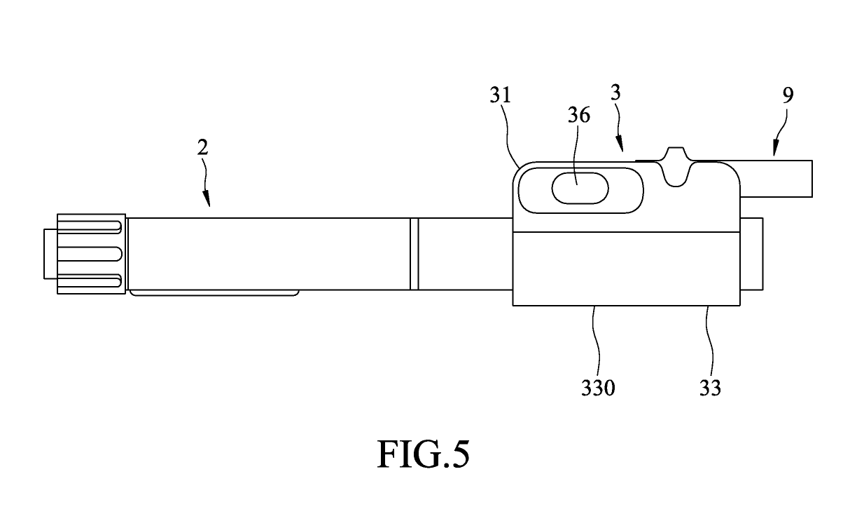

Referring to FIGS. 4 and 5, a second preferred embodiment of the measuring device 3 according to the present invention differs from the first preferred embodiment in that: the positioning component 330 is formed from an elastically deformable material and is connected to the device body 31. The positioning component 330 has a through hole 333 that serves as the receiving space and that permits fitting extension of the drug injection device 2 therethrough by elastic deformation.

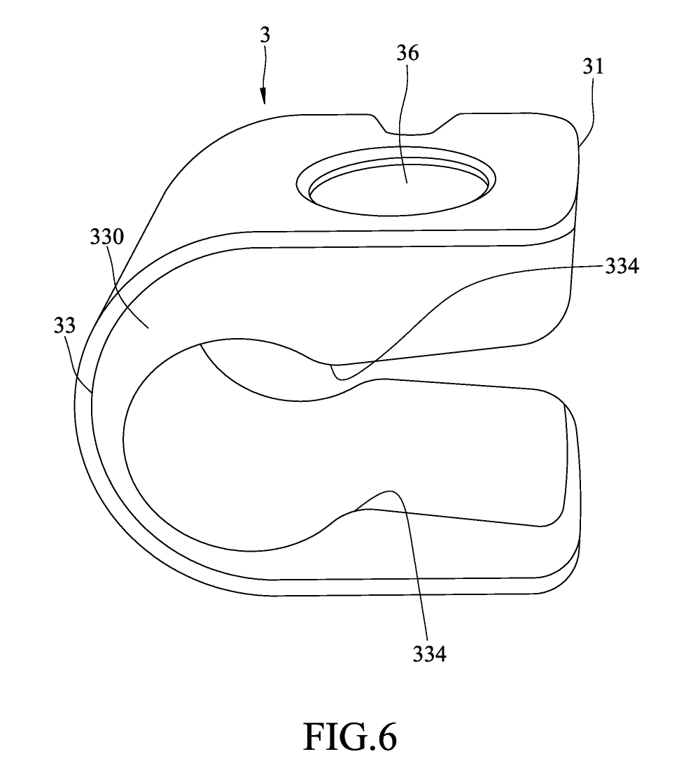

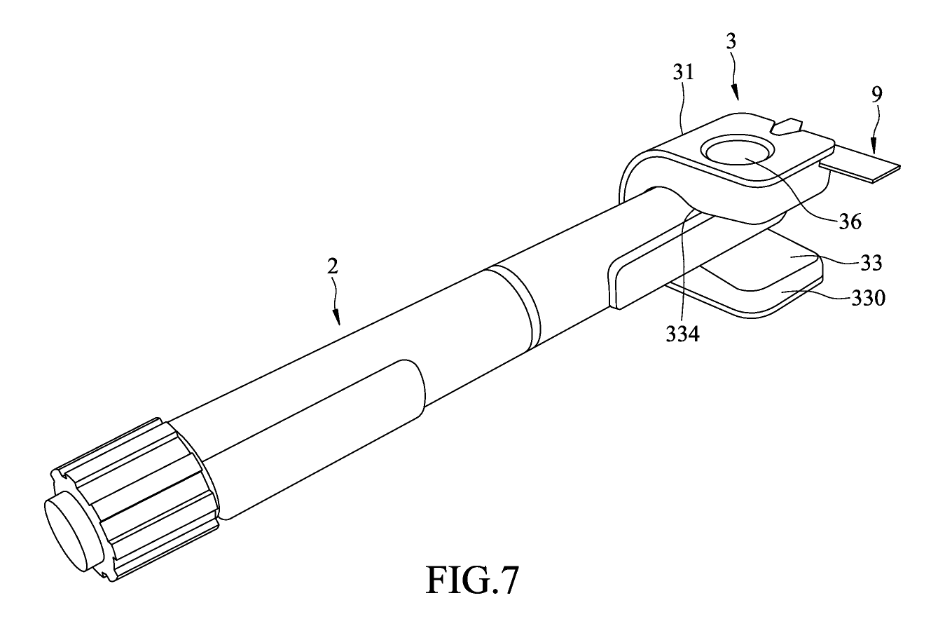

Referring to FIGS. 6 and 7, a third preferred embodiment of the measuring device 3 according to the present invention differs from the first preferred embodiment in that: the device body 31 is of U-shape, and the positioning component 330 is a U-shaped sleeve component that is elastically deformable, that is fixed to the device body 31, that defines the receiving space, and that has a pair of protrusions 334 formed in an inner surface thereof and aligned with each other in a vertical direction for removable retention of the drug injection device 2 in the receiving space. However, the installation unit 33 may include only one protrusion 334, three protrusions 334 or four protrusions 334 in other embodiments, and the present invention should not be limited in this respect.

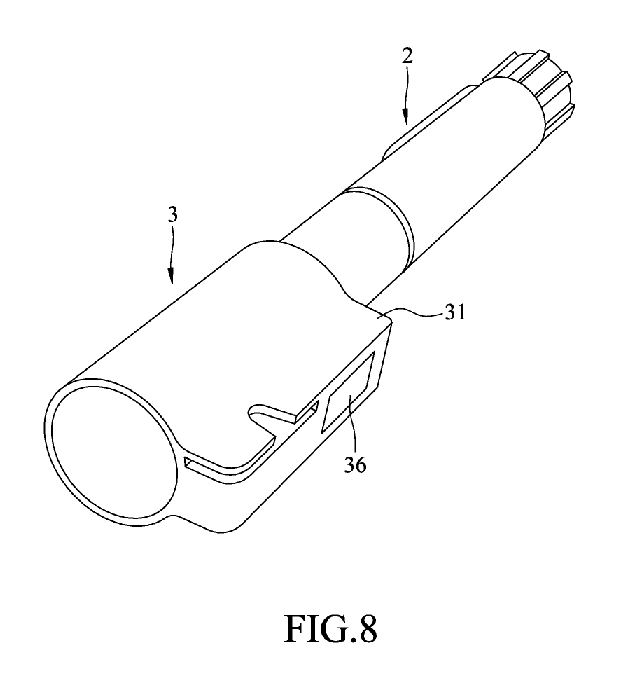

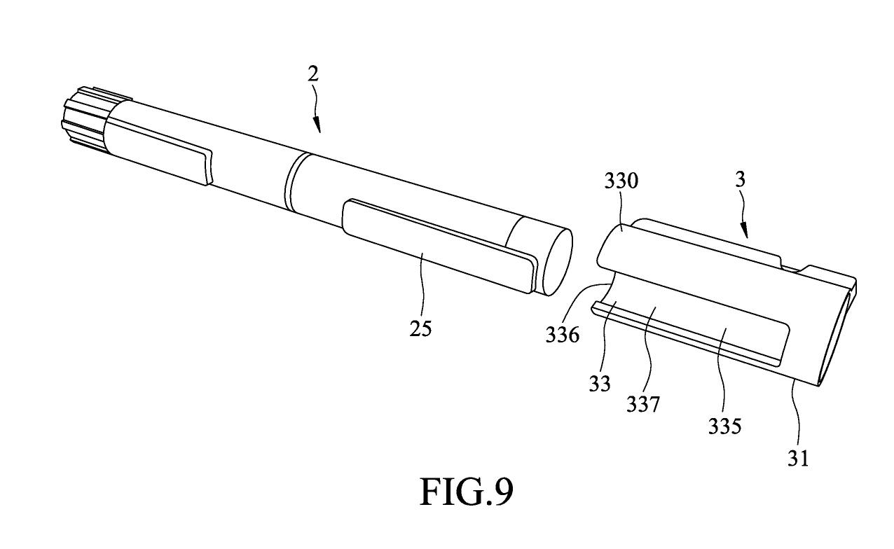

Referring to FIGS. 8 and 9, a fourth preferred embodiment of the measuring device 3 according to the present invention differs from the first preferred embodiment in that: the positioning component 330 is formed on the device body 31, and includes a peripheral wall 335 that surrounds a first axis, that defines the receiving space and an opening 336 in spatial communication with the receiving space, and that is formed with a groove 337 extending along a direction parallel to the first axis and in spatial communication with the opening 336 and the receiving space. The opening 336 permits extension of the drug injection device 2 into the receiving space, and cooperates with the groove 337 to removably position the drug injection device 2 on the device body 31. In detail, the groove 337 is configured in correspondence to a protrusion 25 on an outer peripheral surface of the drug injection device 2, so as to cooperatively enhance engagement and positioning between the installation unit 33 and the drug injection device 2.

While the present invention has been described in connection with what are considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. A measuring device for an analyte in a specimen, comprising:
a device body;
a sensing unit disposed on said device body, and including a measuring module configured to measure a parameter of the analyte in the specimen; and
an installation unit disposed on said device body, and configured to removably install said device body on a drug injection device.

2. The measuring device as claimed in claim 1, wherein said installation unit includes a positioning component disposed on said device body and defining a receiving space, said receiving space being configured to permit extension of the drug injection device therein, so as to retain removably the drug injection device on said positioning component.

3. The measuring device as claimed in claim 2, wherein said positioning component includes a fixing member disposed on an end of said device body, and an encircling member that is elastically deformable and that is disposed on another end of said device body, said encircling member being configured to be removably hooked on said fixing member, and to cooperate with said device body to form said receiving space and to tightly retain the drug injection device on said device body when hooked with said fixing member.

4. The measuring device as claimed in claim 2, wherein said positioning component is formed from an elastically deformable material and is connected to said device body, said positioning component having a through hole that serves as said receiving space and that permits fitting extension of the drug injection device therethrough.

5. The measuring device as claimed in claim 2, wherein said device body is of U-shape, said positioning component being a U-shaped sleeve component that is elastically deformable, that is fixed to said device body, that defines said receiving space, and that has at least one protrusion formed in an inner surface thereof for removable retention of the drug injection device in said receiving space.

6. The measuring device as claimed in claim 2, wherein said positioning component is formed on said device body, and includes a peripheral wall that surrounds a first axis, and that defines said receiving space and an opening in spatial communication with said receiving space, said opening permitting extension of the drug injection device into said receiving space.

7. The measuring device as claimed in claim 6, wherein said positioning component further includes a groove that is formed in said peripheral wall, that extends along a direction parallel to the first axis, and that is in spatial communication with said opening and said receiving space, said groove being configured to cooperate with a protrusion on an outer peripheral surface of the drug injection device to removably position the drug injection device on said device body.

8. The measuring device as claimed in claim 1, wherein said sensing unit further includes a temperature sensing module configured to sense an ambient temperature, and to generate a notification signal when the ambient temperature sensed thereby is out of a preset temperature range.

9. The measuring device as claimed in claim 1, wherein said sensing unit further includes an orientation sensing module configured to generate a notification signal when said installation unit is installed on the drug injection device and a drug output part of the drug injection device is not placed in a prime shot direction that is vertical to a ground plane.

10. The measuring device as claimed in claim 1, further comprising an input unit, a computing unit and a display unit that are disposed on said device body, wherein:
said input unit is operable to input personal data associated with at least one of a height, a weight, a meal time, an amount of carbohydrate, and an amount of exercise;
said computing unit is configured to receive the personal data inputted from said input unit, to perform computation according to the personal data, and to output a computation signal associated with at least one of a recommended dose of a drug, a drug type, and a recommended time of drug injection; and
said display unit is configured to receive the computation signal outputted by said computing unit and to output a display signal corresponding to the computation signal.

11. The measuring device as claimed in claim 1, wherein the analyte is blood glucose, and the drug injection device is an insulin injection device.
